(19) United States
(12) Patent Application Publication
(10) Pub. No.: US 2026/0096759 A1
(43) Pub. Date: Apr. 9, 2026

(54) SYSTEM AND METHOD FOR NEUROMONITORING BASED ON A BIOSIGNAL

(71) Applicant: Dr. Langer Medical GmbH, Waldkirch (DE)

(72) Inventor: Ramona SCHULER, Waldkirch (DE)

(21) Appl. No.: 19/114,904

(22) PCT Filed: Sep. 27, 2022
(86) PCT No.: PCT/EP2022/076872

§ 371 Date: Mar. 25, 2025

(51) Int. Cl. A61B 5/20; A61B 5/00; A61B 5/053; A61B 5/11

(52) U.S. Cl. CPC A61B 5/205; A61B 5/053; A61B 5/1107; A61B 5/4035; A61B 5/4893

(57) ABSTRACT

Provided is a medical system and method for neuromonitoring based on a biosignal. The medical system includes a computing system and performs the method for neuromonitoring based on a biosignal. The method includes monitoring and analysing the biosignal for localizing autonomic nerves associated with a stimulus-induced muscle reaction of smooth muscles of a target organ. The analysing step includes performing time-domain signal analysis of the biosignal to obtain time-domain signal characteristics; performing time-frequency-domain signal analysis of the biosignal to obtain time-frequency-domain signal characteristics; and determining, based on the time-domain signal characteristics and the time-frequency-domain signal characteristics, whether the biosignal is representative of a stimulus-induced muscle reaction. The method further includes outputting an indication that the stimulus-induced muscle reaction has been detected based on determining that the biosignal is representative of the stimulus-induced muscle reaction.

### FIELD OF THE INVENTION

The present invention relates to a method for neuromonitoring based on a baiosignal, a medical system, a computer program product, and a computer-readable medium.

### TECHNICAL BACKGROUND

Surgical interventions in the pelvic region (e.g., low anterior resections in the region of the rectum, urinary bladder, or internal sexual organs) are associated with a high risk of damage to the autonomic and somatic pelvic nerves. The consequences of pelvic nerve damage, particularly of the autonomic nerves, may include postoperative fecal and urinary disorders (bowel and bladder disorders) and sexual dysfunction. This may occur, for example, after low anterior resection (LAR) with total mesorectal excision (TME) for rectal cancer. Thus, since the pelvic nerves provide function to the innervated organs, including the urinary bladder, rectum, and sexual organs, preservation of the nerves is critical to the functional outcome and quality of life of patients.

Accordingly, it is important to reliably localize nerves, particularly including the difficult-to-identify autonomic nerves, so as to avoid damage of said nerves.

The complexity of pelvic neuroanatomy is particularly brough about by the very fine nerve fibers, the predominantly autonomic portions of the nerve plexus, and interindividual differences in nerve location.

Distinguishing the nerves from surrounding tissue, particularly adipose tissue, connective tissue, and scar tissue is very difficult without technical assistance, even with sound knowledge of neuroanatomy and neurophysiology.

The goal of intraoperative neuromonitoring of pelvic nerves is to provide a tool for providing technical support in identifying autonomic pelvic nerves.

The physical principle of nerve identification in currently neuromonitoring is based on direct stimulation of nerves and electrophysiological recording of the response of the target organs. By assessing the stimulation response, conclusions can be made about the course and function of the nerves.

However, neuromonitoring for autonomic nerves presents a challenge because known neuromonitoring techniques for monitoring of the motor and sensory nervous system such as electromyography (EMG) or stimulation and evoked potential (EP) recording cannot be directly transferred to autonomic nerves because of the differences in excitation and stimulus-response.

Specifically, the striated muscles of the motor unit contract with a latency of a few milliseconds after application of a stimulation pulse to the innervating nerve. The refractory period is a few milliseconds. In contrast, excitation of the autonomic nervous system does not lead to stimulation pulse synchronous muscle action potentials. Accordingly, the presently known techniques that are suitable for the motor and sensory nervous system is not readily applicable to autonomic nerves.

The present invention has the object of providing a method, medical system, computer program product, and computer-readable medium that allow for overcoming at least some of the above-identified challenges.

In particular, it is an object of the present invention to provide techniques or tools for reliably localizing autonomic nerves.

Aspects of the present invention, examples and exemplary steps and their embodiments are disclosed in the following. Different exemplary features of the invention can be combined in accordance with the invention wherever technically expedient and feasible.

### EXEMPLARY SHORT DESCRIPTION OF THE INVENTION

In the following, a short description of the specific features of the present invention is given which shall not be understood to limit the invention only to the features or a combination of the features described in this section.

The invention provides a method, a medical system, a computer program product, and a computer-readable medium according to the independent claims. Preferred embodiments are laid down in the dependent claims.

The present disclosure provides, among others, a method for neuromonitoring based on a biosignal, the method comprising monitoring and analysing a biosignal so as to localize autonomic nerves associated with a stimulus-induced muscle reaction of smooth muscles of a target organ. The method comprises signal analysis of a biosignal to obtain time-domain signal characteristics and time-frequency-domain signal characteristics and, based thereon, determining whether the biosignal is representative of a stimulus-induced muscle reaction of the smooth muscles of a target organ.

### GENERAL DESCRIPTION OF THE INVENTION

In this section, a description of the general features of the present invention is given for example by referring to possible embodiments of the invention.

The present disclosure provides a method for neuromonitoring based on a biosignal, the method comprising monitoring and analysing a biosignal so as to localize autonomic nerves associated with a stimulus-induced muscle reaction of smooth muscles of a target organ.

More specifically, the analysing of the biosignal may, for example, entail analysing the shape of the biosignal. For example, the biosignal may be analysed to detect shapes characteristic for stimulus-induced muscle reactions of smooth muscles.

The method may allow for localizing autonomic nerves by means of nerve stimulation of the autonomic nerves, i.e., by applying a stimulus to a tissue at a target location. The nerve stimulation causes the stimulus-induced muscle reaction of the smooth muscles. Accordingly, when analysing the biosignal in such a manner as to detect a stimulus-induced muscle reaction, it is possible to determine whether nerve stimulation of the autonomic nerves has occurred when applying the stimulus to the tissue at said target location and, accordingly, to localize autonomic nerves. In particular, the method may comprise distinguishing whether features in the biosignal are representative of stimulus-induced muscle reaction or stem from artifacts and other muscle activity or movement, e.g., caused by organ movements and/or respiration,

The stimulus may, for example, be an electric stimulus, e.g., having a square wave signal. It may be selectively applied to the tissue by a monopolar or bipolar electrode. The electric stimulus may, in particular, be applied to the tissue by means of a handheld probe.

As will be seen in more detail below, the stimulus-induced muscle reaction may, for example, be a stimulus-induced muscle contraction. The target organ may, for example, be the rectum or the bladder.

The biosignal may, for example, be an impedance signal obtained from measuring an impedance of the muscles. Alternatively, the biosignal may, for example, be a bladder pressure signal obtained by measuring the bladder pressure.

It is to be understood that the present method may be carried out for several different biosignals, e.g., a first and a second biosignal, at the same time. For example, the first and second biosignal may be a bladder pressure signal and an impedance signal, respectively, or may be different impedance signals, e.g., impedance of the rectum and impedance of the bladder.

When carrying out the method for different biosignals, optionally, the same stimulus may be used. Acquisition may be performed separately for each of the different biosignals. Each of the method steps described in the present disclosure for the biosignal may, accordingly, be performed for each of the different (e.g., first and second) biosignals. This may yield improved reliability.

According to the present disclosure, the method, particularly the analysing, comprises performing time-domain signal analysis of the biosignal to obtain one or more time-domain signal characteristics, the biosignal based on measurement data obtained by a neuromonitoring device.

The biosignal being based on measurement data obtained by a neuromonitoring device is to be understood broadly. This may entail that the biosignal corresponds to the signal as output by the neuromonitoring device. Alternatively, the biosignal may be obtained by pre-processing output data from the neuromonitoring device, e.g., filtering and/or normalizing output data from the neuromonitoring device. More details are outlined below. Measurement data output by the neuromonitoring device may be impedance measurement data from measuring the impedance of the smooth muscles or bladder pressure measurement data.

The time-domain signal analysis is to be understood broadly as an analysis of the biosignal in the time-domain. Time-domain signal characteristics are to be understood broadly and may contain any characteristics derivable from the biosignal in the time domain. They may comprise characteristics derived from the biosignal itself and/or from a derivative of the biosignal and/or from integration of the biosignal. A more detailed description will follow.

According to the present disclosure, the method, particularly the analysing, comprises performing time-frequency-domain signal analysis of the biosignal to obtain one or more time-frequency-domain signal characteristics.

The time-frequency-domain signal analysis is to be understood broadly as an analysis of the biosignal in the time-frequency-domain. Time-frequency-domain signal characteristics are to be understood broadly and may contain any characteristics derivable from the biosignal in the time-frequency domain. They may comprise characteristics derived from a time-frequency transformation of the biosignal itself and/or from a time-frequency transformation of a derivative of the biosignal. For example, transformation coefficients of the time-frequency transformation, particularly their magnitude, may be analysed as part of the time-frequency-domain signal analysis. For example, Wavelet Transform, WT, particularly Continuous Wavelet Transform, CWT, or Discrete Wavelet Transform, DWT, or Short-Time Fourier Transform, STFT, may be employed for the time-frequency transformation, and their respective transformation coefficients may be analysed as part of the time-frequency-domain signal analysis.

According to the present disclosure, the method, particularly the analysing, comprises, based on the time-domain signal characteristics and time-frequency-domain signal characteristics determining whether the biosignal is representative of a stimulus-induced muscle reaction.

For example, criteria may be associated with the shape of the biosignal in the time domain, e.g., a change thereof from a base level, onset of the change, maximum amplitude of the change, time from onset of the change to reaching maximum amplitude of the change, or the like.

A change in the biosignal may represent a feature of the biosignal representative of a stimulus-induced muscle reaction of smooth muscles or it may be due to artifacts or other muscle activity.

Analysing the biosignal may involve determining candidate features, e.g., in the time-domain and/or in the time-frequency domain, and determining, which among the candidate features are features representative of a stimulus-induced muscle reaction of smooth muscles. Thus, artifacts and non-stimulus-induced muscle activity may be distinguished from stimulus-induced muscle reactions of smooth muscles. This will be explained in more detail below.

According to the present disclosure, the method comprises, in case it is determined that the biosignal is representative of a stimulus-induced muscle reaction, outputting an indication that a stimulus-induced muscle reaction has been detected.

In other words, an output may be provided that indicates that a nerve whose stimulation causes a stimulus-induced muscle reaction of smooth muscles of the target organ has been detected at the location where the stimulus was applied.

As can be seen from the above, autonomic nerves can be localized by means of the method of the present disclosure.

Thus, to summarize, the method of the present disclosure allows for reliably localizing autonomic nerves.

Contrary to the motor nervous system, stimulation of autonomic nerves generally may lead to a modulation of smooth muscle activity by triggering action potential volleys (spikes). Smooth muscle activity is characterized by rhythmic spontaneous changes in membrane potential, referred to as slow waves, e.g., with a frequency of 3-15/min. Only when the slow waves exceed a threshold potential, spikes are triggered, resulting in a sluggish reaction, particularly contraction, of the smooth muscles to a stimulus. This slow smooth muscle reaction, e.g., contraction, usually occurs several seconds after stimulation pulses are applied to the tissue. Accordingly, the timing of the stimulus alone is not sufficient for reliably associating a biosignal change with the stimulus, which makes the method of the present disclosure all the more advantageous.

Obtaining measurement data on which the biosignal is based may involve impedance measurements, such that the biosignal may be an impedance signal. More specifically, for example, impedance measurements on pelvic smooth muscle during direct stimulation of tissue that may comprise pelvic autonomic nerves may allow for localization of pelvic autonomic nerves.

For example, the neuromonitoring device may comprise an impedance measurement device or bladder pressure measurement device.

As will be seen below, a direct nerve stimulator configured to apply a stimulus may be employed in the method of present disclosure. Alternatively or in addition, a display device configured to output an indication that a stimulus-induced muscle reaction has been detected may be employed in the method of present disclosure.

As an example, according to the present disclosure, at least two electrodes for impedance measurement may be placed per organ (e.g., urinary bladder and/or rectum), and direct nerve stimulation may be performed with a handheld probe. With this setup and the method of the present disclosure, it is possible to measure stimulus-induced (slow) smooth muscle reaction, e.g., contractions.

It is noted that the present disclosure provides a method that may also allow for automatically distinguishing biosignals representative of stimulus-induced muscle reaction from signal features caused by organ movements or respiration. While they do differ in signal shape and their frequency, reliably distinguishing them is very challenging, particularly visually distinguishing them by a human operator, which requires skill, causes delays due to the reviewing process, and is quite error prone. The signal analysis of the present disclosure allows for overcoming these challenges.

Thus, the present disclosure provides a method that addresses at least some of the above-described challenges.

According to the present disclosure the method may comprise, based on the time-domain signal characteristics and time-frequency-domain signal characteristics, distinguishing between features in the biosignal being representative of artifacts and features in the biosignal being representative of a stimulus-induced muscle reaction. Optionally, the distinguishing may additionally comprise distinguishing features in the biosignal being representative of a stimulus-induced muscle reaction from features in the biosignal that are not representative of a significant biosignal response, in particular a significant impedance change or a significant bladder pressure change. The tissue impedance may refer to a tissue impedance.

That is, the method may comprise identifying features in the biosignal and classifying said features into a plurality of classes, one class being features representative of a stimulus-induced muscle reaction and another class being features representative of artifacts.

According to the present disclosure, the time-domain signal characteristics may comprise a maximum amplitude within the biosignal waveform, in particular an impedance change or a bladder pressure change, relative to a base-level of the biosignal, in particular, an impedance base-level or bladder pressure base-level.

A base-level of the biosignal may be an initial state of the biosignal waveform prior to a transient signal characteristic or signal change, e.g., prior to muscle reaction.

Alternatively or in addition, the time-domain signal characteristics may comprise an onset latency of the transient signal change within the biosignal waveform. The onset latency may be the time of an onset of a biosignal change after application of a stimulus. To determine the onset latency, data on the applied stimulus may be used as input, for example data retrieved from a data storage or received from a device applying the stimulus, e.g., in real time.

Alternatively or in addition, the time-domain signal characteristics may comprise a gradient of the transient signal change within the biosignal and/or a time to reach maximum gradient of the transient signal change within the biosignal. Alternatively or in addition, the time-domain signal characteristics may comprise a duration of a transient signal change within the biosignal from an onset of the transient signal change, and/or regression coefficients, e.g., linear, polynomial, or cubic spline.

According to the present disclosure, the time-frequency-domain signal characteristics may comprise a magnitude of transformation coefficients, in particular, a localization of magnitude extrema of transformation coefficients and/or absolute or relative value of the magnitude extrema. The features in the time-frequency domain mentioned above may comprise magnitude extrema. As explained in the context of said features, they may be distinguished, i.e., classified, as being representative of a stimulus-induced muscle reaction or artifacts or non-significant biosignal changes.

As an example, only extrema in certain time-frequency regions and/or extrema having at least a predetermined minimum distance from neighbouring extrema and/or extrema having at least a predetermined minimum value of the magnitude may be classified as representative of a stimulus-induced muscle reaction.

According to the present disclosure, the time-frequency-domain signal analysis may comprise transforming the biosignal or the first derivative of the biosignal to the time-frequency domain and analysing one or more transformation coefficients, particularly a localization of magnitude extrema of the transformation coefficients and/or absolute value and/or relative value of the magnitude extrema of the transformation coefficients.

According to the present disclosure, the time-frequency-domain signal analysis may comprise an analysis of transformation coefficients of a wavelet transform, WT, e.g., a CWT or DWT, or an analysis of transformation coefficients of a STFT.

The time-frequency-domain signal analysis may, in particular, comprise selecting a window function and scaling the window function, e.g., stretching and/or compressing the window function and/or shifting the window function. The time-frequency-domain signal analysis may further comprise obtaining, for each of a plurality of samples, one or more transformation coefficients. The time-frequency-domain signal analysis may further comprise analysing at least a subset of the one or more coefficients, in particular, analysing a magnitude of the one or more coefficients as a function of the scaling factors and/or the shifting factors. As an example, the time-frequency-domain signal analysis may comprise analysing said coefficients as described above, e.g., by analysing the extrema of the magnitude of the coefficients.

Samples, in the present disclosure, may be analog to digital converted instantaneous measurement values acquired by a neuromonitoring device configured to acquire measurement data, in particular an impedance measurement device and/or pressure measurement device.

According to the present disclosure, analysing the one or more transformation coefficients may comprise representing the magnitude of one or more of the transformation coefficients as a function of time and frequency, in particular representing CWT, DWT, or STFT coefficients as a function of shifting and/or scaling factors.

Analysing the one or more transformation coefficients may further comprise identifying candidate features in the function, in particular, identifying local maxima and/or minima of the function as candidate features.

Analysing the one or more transformation coefficients may further comprise determining, for at least one of the candidate features, whether the candidate feature is representative of a stimulus-induced muscle reaction, the determining based on one or more of the time-domain signal characteristics and/or based on one or more of the time-frequency-domain signal characteristics, in particular, position, i.e. time and frequency/wavelet scale, and/or magnitude of the candidate feature.

For example, candidate features having a magnitude below a predetermined threshold (magnitude-based threshold) may be discarded. Alternatively or in addition, candidate features having a distance in time below a predetermined threshold (time based threshold), particularly overlapping candidate features, may be discarded, wherein the candidate feature having the maximum magnitude within the overlapping candidate features, may be retained as the resultant candidate feature.

Alternatively or in addition, the wavelet scale corresponding to the resultant candidate feature may be the primary criterion to distinguish between stimulus-induced muscle reactions and artifacts. Candidate features representative of artifacts may be separated from candidate features representative of stimulus-induced muscle reactions based on a predetermined threshold wavelet scale. Resultant candidate features with corresponding wavelet scale below the predetermined threshold wavelet scale and resultant candidate features with corresponding wavelet scale above the predetermined threshold wavelet scale, that fall within a predetermined time interval, may be compared in magnitude. The resultant candidate features with corresponding wavelet scale below the predetermined threshold wavelet scale and with corresponding magnitude below the magnitude of resultant candidate features with corresponding wavelet scale above the predetermined threshold wavelet scale may be discarded.

According to the present disclosure, the time-frequency-domain signal analysis may comprise analysing the one or more transformation coefficients taking into account the time-domain signal characteristics and/or information derived from the time-domain signal characteristics. In particular, determining whether a candidate feature is representative of a stimulus-induced muscle reaction may be performed taking into account the time-domain signal characteristics and/or the information derived from the time-domain signal characteristics.

For example, candidate features obtained by time-frequency domain signal analysis may be discarded based on time-domain signal characteristics, e.g., in case it is determined that the time-domain signal characteristics preclude that there is a feature representative of a stimulus-induced muscle reaction where the candidate feature was identified in the time-frequency domain.

Thus, improved reliability may be obtained, particular in terms of false positives.

According to the present disclosure, the time-frequency-domain signal analysis may comprise determining a time frame corresponding to a selected portion of the time-domain biosignal, in particular a portion after the onset of an/the biosignal change. In particular, the portion may be selected based on a gradient of the time-domain biosignal.

The time-frequency-domain signal analysis may comprise determining whether a candidate feature is representative of a stimulus-induced muscle reaction at least based on the determined time frame. In particular, the candidate feature being within the time frame may be one of one or more criteria for determining whether a candidate feature is representative of a stimulus-induced muscle reaction. For example, a candidate feature being outside of the time frame may lead to discarding the candidate feature. Thus, it is possible to quickly discard any candidate features that very clearly cannot be representative of a stimulus-induced muscle reaction, e.g. because it is too early to expect a stimulus-induced muscle reaction.

The method of the present disclosure may comprise performing a Continuous Wavelet Transform, CWT, of the biosignal or of the first derivative of the biosignal, particularly a first derivative of an/the impedance signal. Alternatively, the method of the present disclosure may comprise performing a Discrete Wavelet Transform, DWT, of the biosignal or of the first derivative of the biosignal, particularly a first derivative of an/the impedance signal. Alternatively, the method of the present disclosure may comprise performing a Short-Time Fourier Transform, STFT of the biosignal or the first derivative of the biosignal, particularly a first derivative of an/the impedance signal.

These different transforms each yield transformation coefficients suitable for the above-described analysis and also provide a resolution that allows for particularly reliable determination of whether the biosignal is representative of a stimulus-induced muscle reaction.

According to the present disclosure, the monitoring and/or the analysing of the biosignal may be performed triggered by receiving an indication of a stimulus being applied to a tissue. As an example, monitoring of the biosignal may be performed intermittently, e.g., only at times when a stimulus-induced muscle reaction is to be expected. As an example, monitoring may be triggered by data indicating that a stimulus has been or will be applied. Such data may be received from the device used for applying the stimulus, for example.

According to the present disclosure, time-domain signal analysis and/or time-frequency domain signal analysis may be performed taking into account one or more characteristics of the stimulus, particularly onset time, length, and/or amplitude of the stimulus. The characteristics of the stimulus may be determined from data received from a device used for applying the stimulus and/or may be stored data. Characteristics of the stimulus may comprise and/or be derived from operation parameter settings of the device used for applying the stimulus, for example.

The method of the present disclosure may comprise pre-processing an output signal of the neuromonitoring device to obtain the biosignal, the pre-processing comprising normalizing the output signal with respect to a base-level of the biosignal, in particular base-level impedance or base-level bladder pressure, and/or applying a low pass filter, particularly after normalizing the output signal, and/or performing a sweep extraction. The impedance may refer to a tissue impedance.

As mentioned above, a base-level of the biosignal may be an initial state of the biosignal waveform prior to a transient signal characteristic or signal change, e.g., prior to muscle reaction. For example, when the biosignal is an impedance signal, a base-level impedance may be the level of the impedance signal in an initial state of the impedance signal waveform prior to a transient signal characteristic or signal change, e.g., prior to muscle reaction. The impedance signal may be representative not only of the impedance of the muscle, but also the device, the leads, the electrodes, other tissues, and the like.

Using the example of an impedance, which may similarly apply to a bladder pressure signal, normalization may be performed in such a manner that the normalized signal is U(t)/U(0), U(t) being the output signal, e.g. measured impedance or bladder pressure, at time t and U(0) the measured base-level of the output signal, e.g., the base-level impedance or base-level bladder pressure. Thus, a dimensionless signal that is proportional to the actual change, e.g., impedance change in the tissue or bladder pressure change, may be obtained.

As mentioned above, pre-processing may also comprise filtering. As an example, a low pass filter may be applied to an output signal of a neuromonitoring device or a normalized output signal. Such a filter may remove or reduce artifacts, e.g., in case of impedance measurements artifacts from a change in membrane potential, noise, or breathing. It may result in a smoother signal.

A filter that is shape-preserving may be employed for the filtering. Thus, a characteristic shape of the biosignal, e.g., impedance or bladder pressure change, as caused by a stimulus-induced muscle reaction is preserved, allowing for improved signal analysis. A digital filter, for example an IIR filter, may be employed.

The method of the present disclosure may comprise integration and/or differentiation, e.g., determining a first derivate, of the biosignal, wherein the results of the integration and/or differentiation may be used as input for the time-domain signal analysis and/or for the time-frequency domain signal analysis.

For example, a first derivative may be transformed to the time-frequency domain for time-frequency signal analysis. This allows for specifically analysing the signal based on its shape, e.g., to identify whether a shape that is characteristic of a stimulus-induced muscle reaction is present.

Alternatively, the (original) biosignal may be transformed to the time-frequency domain for time-frequency signal analysis, utilizing the expected transient signal change waveform as window function. This allows for specifically analysing the signal based on its shape, e.g., to identify whether a shape that is characteristic of a stimulus-induced muscle reaction is present.

According to the present disclosure, the time-domain signal analysis and time-frequency domain signal analysis, and optionally integration and/or differentiation of the biosignal, may be performed continuously or for each of a plurality of sweeps of the biosignal, particularly successive sweeps of the biosignal.

In case the time-domain signal analysis and time-frequency domain signal analysis, and optionally integration and/or differentiation of the biosignal, are performed for each of a plurality of sweeps of the biosignal, particularly successive sweeps of the biosignal, time-domain signal characteristics and time-frequency-domain signal characteristics may be determined individually and optionally independently for each sweep. In this case, optionally some or all of the pre-processing steps may also be performed separately and optionally independently for each sweep.

A sweep is to be understood broadly and may be a portion of the signal, e.g., each sweep having a predetermined duration.

According to the present disclosure, outputting an indication that a stimulus-induced muscle reaction has been detected may comprise providing an acoustic and/or visual and/or haptic output to a user. For example, a warning may be displayed on a display device, e.g., as part of a GUI, and/or a visual, haptic and/or acoustic warning signal may be output. Any suitable means may be used for this purpose, e.g., known indicator devices.

According to the present disclosure, as mentioned above, the biosignal may be an impedance signal or a bladder pressure signal.

According to the present disclosure, as mentioned above, the stimulus-induced muscle reaction may be a muscle contraction.

The method of the present disclosure may comprise acquiring measurement data by means of a neuromonitoring device, particularly continuously. In particular, the method of the present disclosure may comprise measuring, particularly continuously, an impedance of smooth muscles of a target organ by means of an impedance measurement device. Alternatively or in addition, the method of the present disclosure may comprise measuring, particularly continuously, a bladder pressure by means of a pressure measurement device.

The method of the present disclosure may comprise applying, particularly by means of a hand-held device, a stimulus to a portion of a tissue. The stimulus may be a square wave signal, for example. The method may also optionally comprise providing information on the characteristics of the stimulus, e.g., pulse length, amplitude, or the like, as an input for the analysis of the biosignal. Alternatively or in addition, the method may comprise outputting a trigger signal to trigger monitoring of the biosignal, for example prior to or upon applying the stimulus.

The method of the present disclosure may comprise, in response to determining that the biosignal is representative of a stimulus-induced muscle reaction caused by the stimulus applied to the portion of the tissue, outputting an indication that the portion of the tissue comprises nerves associated with the smooth muscles.

Outputting an indication that a stimulus-induced muscle reaction has been detected and/or outputting an indication that the portion of the tissue comprises nerves associated with the smooth muscles may be performed immediately upon determining that the biosignal is representative of a stimulus-induced muscle reaction.

That is, output may inform a user in near-real time when a stimulus-induced muscle reaction has occurred and, accordingly, when the applied stimulus stimulated autonomic nerves.

According to the present disclosure, the method steps may be performed for at least two biosignals, the biosignals obtained for different target organs, so as to localize autonomic nerves associated with a stimulus-induced muscle reaction of smooth muscles of each of the different target organs.

For example, according to the present disclosure, the method may comprise acquiring measurement data, e.g., impedance data, for at least two different organs. For example, the impedance of the bladder and the impedance of the rectum may be acquired. Thus, at least two biosignals may be obtained, e.g., one based on the measured impedance of the bladder and another one based on the measured impedance of the rectum. The method steps of the present disclosure that are carried out subsequent to acquiring the measurement data for the different target organs may be performed for each of these biosignals.

This may not require applying separate stimuli to the tissue. In particular, a common stimulus may be applied and each of two or more biosignals may be analysed to determine whether it is representative of a stimulus-induced muscle reaction.

Some or all of the steps of monitoring and analysing the biosignal so as to localize autonomic nerves associated with a stimulus-induced muscle reaction of smooth muscles of a target organ according to the present disclosure, particularly all steps of the methods according to the present disclosure, may be performed automatically.

The present disclosure also provides a medical system configured to carry out the method of the present disclosure, that is one or more, in particular all of the steps of the method of the present disclosure.

In particular, the medical system may comprise a computing system configured to carry out and/or control one or more, in particular all of the steps of the method of the present disclosure. The computing system may comprise a processing means configured to perform the monitoring and analysing of the biosignal.

The computing system may also comprise one or more output devices, e.g., a display device or other visual output device and/or an audio output device and/or a haptic output device, e.g., a vibration generating device, the output devices configured to perform the step of outputting an indication that a stimulus-induced muscle reaction has been detected. As an example, a suitable signalling device may be used for performing the step of outputting the indication.

The medical system of the present disclosure may comprise a, particularly hand-held, device configured to apply a stimulus to a portion of a tissue, e.g., a handheld probe. For example, the device may comprise an instrument consisting of a handpiece and an anode and/or a cathode for transmission of the stimulus from the nerve stimulator device to a portion of the tissue.

Alternatively or in addition, the medical system of the present disclosure may comprise a neuromonitoring device configured to acquire the measurement data, in particular, an impedance measurement device configured to measure, particularly continuously, an impedance of smooth muscles of a target organ or a pressure measurement device configured to measure, particularly continuously, a bladder pressure.

As mentioned above, the present disclosure may comprise acquiring measurement data, e.g., impedance data, for at least two different organs. Accordingly, the medical system may be configured to acquire measurement data for at least two target organs, e.g., by a separate neuromonitoring device or by a common neuromonitoring device. Particularly, the medical system may be configured such that the impedance of the bladder and the impedance of the rectum may be acquired. Thus, at least two biosignals may be obtained. The method steps of the present disclosure that are carried out subsequent to acquiring the measurement data for the different target organs may be performed for each of the biosignals.

As an example, the neuromonitoring device may comprise at least a pair of electrodes configured to be attached to each target organ for measuring an impedance.

The present disclosure also provides a computer program product comprising instructions which, when the program is executed by a computer, cause the computer to carry out and/or control the method of the present disclosure, that is one or more, in particular all of the steps of the method of the present disclosure.

The present disclosure also provides a computer-readable medium comprising instructions which, when executed by a computer, cause the computer to carry out and/or control the method of the present disclosure, that is one or more, in particular all of the steps of the method of the present disclosure.

For the sake of completeness, it is noted that the invention does not involve or in particular comprise or encompass an invasive step which would represent a substantial physical interference with the body requiring professional medical expertise to be carried out and entailing a substantial health risk even when carried out with the required professional care and expertise. For example, the invention does not comprise a step of positioning a medical implant in order to fasten it to an anatomical structure or a step of fastening the medical implant to the anatomical structure or a step of preparing the anatomical structure for having the medical implant fastened to it. More particularly, the invention does not involve or in particular comprise or encompass any surgical or therapeutic activity. The invention is instead directed as applicable to localization of nerves at a given tissue portion. For this reason alone, no surgical or therapeutic activity and in particular no surgical or therapeutic step is necessitated or implied by carrying out the invention.

The features and advantages outlined above in the context of the method similarly apply to the medical system, the computer program product, and the computer readable medium of the present disclosure.

The present disclosure also relates to the use of the medical system or any embodiment thereof for localization of autonomic nerves. The use comprises for example investigating whether a selected portion of a tissue comprises the autonomic nerves.

### DEFINITIONS

In this section, definitions for specific terminology used in this disclosure are offered which also form part of the present disclosure.

### Computer Implemented Method

The method in accordance with the invention is for example a computer implemented method. For example, all the steps or merely some of the steps (i.e. less than the total number of steps) of the method in accordance with the invention can be executed by a computer (for example, at least one computer). An embodiment of the computer implemented method is a use of the computer for performing a data processing method. An embodiment of the computer implemented method is a method concerning the operation of the computer such that the computer is operated to perform one, more or all steps of the method.

The computer for example comprises at least one processor and for example at least one memory in order to (technically) process the data, for example electronically and/or optically. The processor being for example made of a substance or composition which is a semiconductor, for example at least partly n-and/or p-doped semiconductor, for example at least one of II-, III-, IV-, V-, VI-semiconductor material, for example (doped) silicon and/or gallium arsenide. The calculating or determining steps described are for example performed by a computer. Determining steps or calculating steps are for example steps of determining data within the framework of the technical method, for example within the framework of a program. A computer is for example any kind of data processing device, for example electronic data processing device. A computer can be a device which is generally thought of as such, for example desktop PCs, notebooks, netbooks, etc., but can also be any programmable apparatus, such as for example a mobile phone or an embedded processor. A computer can for example comprise a system (network) of “sub-computers”, wherein each sub-computer represents a computer in its own right. The term “computer” includes a cloud computer, for example a cloud server. The term “cloud computer” includes a cloud computer system which for example comprises a system of at least one cloud computer and for example a plurality of operatively interconnected cloud computers such as a server farm. Such a cloud computer is preferably connected to a wide area network such as the world wide web (WWW) and located in a so-called cloud of computers which are all connected to the world wide web. Such an infrastructure is used for “cloud computing”, which describes computation, software, data access and storage services which do not require the end user to know the physical location and/or configuration of the computer delivering a specific service. For example, the term “cloud” is used in this respect as a metaphor for the Internet (world wide web). For example, the cloud provides computing infrastructure as a service (laaS). The cloud computer can function as a virtual host for an operating system and/or data processing application which is used to execute the method of the invention. The cloud computer is for example an elastic compute cloud (EC2) as provided by Amazon Web Services™. A computer for example comprises interfaces in order to receive or output data and/or perform an analogue-to-digital conversion. The data are for example data which represent physical properties and/or which are generated from technical signals. The technical signals are for example generated by means of (technical) detection devices (such as for example devices for detecting marker devices) and/or (technical) analytical devices (such as for example devices for performing (medical) imaging methods), wherein the technical signals are for example electrical or optical signals. The technical signals for example represent the data received or outputted by the computer. The computer is preferably operatively coupled to a display device which allows information outputted by the computer to be displayed, for example to a user. One example of a display device is a virtual reality device or an augmented reality device (also referred to as virtual reality glasses or augmented reality glasses) which can be used as “goggles” for navigating. A specific example of such augmented reality glasses is Google Glass (a trademark of Google, Inc.). An augmented reality device or a virtual reality device can be used both to input information into the computer by user interaction and to display information outputted by the computer. Another example of a display device would be a standard computer monitor comprising for example a liquid crystal display operatively coupled to the computer for receiving display control data from the computer for generating signals used to display image information content on the display device. A specific embodiment of such a computer monitor is a digital lightbox. An example of such a digital lightbox is Buzz®, a product of Brainlab AG. The monitor may also be the monitor of a portable, for example handheld, device such as a smart phone or personal digital assistant or digital media player.

The invention also relates to a program which, when running on a computer, causes the computer to perform one or more or all of the method steps described herein and/or to a program storage medium on which the program is stored (in particular in a non-transitory form) and/or to a computer comprising said program storage medium and/or to a (physical, for example electrical, for example technically generated) signal wave, for example a digital signal wave, carrying information which represents the program, for example the aforementioned program, which for example comprises code means which are adapted to perform any or all of the method steps described herein.

Within the framework of the invention, computer program elements can be embodied by hardware and/or software (this includes firmware, resident software, micro-code, etc.). Within the framework of the invention, computer program elements can take the form of a computer program product which can be embodied by a computer-usable, for example computer-readable data storage medium comprising computer-usable, for example computer-readable program instructions, “code” or a “computer program” embodied in said data storage medium for use on or in connection with the instruction-executing system. Such a system can be a computer; a computer can be a data processing device comprising means for executing the computer program elements and/or the program in accordance with the invention, for example a data processing device comprising a digital processor (central processing unit or CPU) which executes the computer program elements, and optionally a volatile memory (for example a random access memory or RAM) for storing data used for and/or produced by executing the computer program elements. Within the framework of the present invention, a computer-usable, for example computer-readable data storage medium can be any data storage medium which can include, store, communicate, propagate or transport the program for use on or in connection with the instruction-executing system, apparatus or device. The computer-usable, for example computer-readable data storage medium can for example be, but is not limited to, an electronic, magnetic, optical, electromagnetic, infrared or semiconductor system, apparatus or device or a medium of propagation such as for example the Internet. The computer-usable or computer-readable data storage medium could even for example be paper or another suitable medium onto which the program is printed, since the program could be electronically captured, for example by optically scanning the paper or other suitable medium, and then compiled, interpreted or otherwise processed in a suitable manner. The data storage medium is preferably a non-volatile data storage medium. The computer program product and any software and/or hardware described here form the various means for performing the functions of the invention in the example embodiments. The computer and/or data processing device can for example include a guidance information device which includes means for outputting guidance information. The guidance information can be outputted, for example to a user, visually by a visual indicating means (for example, a monitor and/or a lamp) and/or acoustically by an acoustic indicating means (for example, a loudspeaker and/or a digital speech output device) and/or tactilely by a tactile indicating means (for example, a vibrating element or a vibration element incorporated into an instrument). For the purpose of this document, a computer is a technical computer which for example comprises technical, for example tangible components, for example mechanical and/or electronic components. Any device mentioned as such in this document is a technical and for example tangible device.

### Acquiring Data

The expression “acquiring data” for example encompasses (within the framework of a computer implemented method) the scenario in which the data are determined by the computer implemented method or program. Determining data for example encompasses measuring physical quantities and transforming the measured values into data, for example digital data, and/or computing (and e.g. outputting) the data by means of a computer and for example within the framework of the method in accordance with the invention. The meaning of “acquiring data” also for example encompasses the scenario in which the data are received or retrieved by (e.g. input to) the computer implemented method or program, for example from another program, a previous method step or a data storage medium, for example for further processing by the computer implemented method or program. Generation of the data to be acquired may but need not be part of the method in accordance with the invention. The expression “acquiring data” can therefore also for example mean waiting to receive data and/or receiving the data. The received data can for example be inputted via an interface. The expression “acquiring data” can also mean that the computer implemented method or program performs steps in order to (actively) receive or retrieve the data from a data source, for instance a data storage medium (such as for example a ROM, RAM, database, hard drive, etc.), or via the interface (for instance, from another computer or a network). The data acquired by the disclosed method or device, respectively, may be acquired from a database located in a data storage device which is operably to a computer for data transfer between the database and the computer, for example from the database to the computer. The computer acquires the data for use as an input for steps of determining data. The determined data can be output again to the same or another database to be stored for later use. The database or database used for implementing the disclosed method can be located on network data storage device or a network server (for example, a cloud data storage device or a cloud server) or a local data storage device (such as a mass storage device operably connected to at least one computer executing the disclosed method). The data can be made “ready for use” by performing an additional step before the acquiring step. In accordance with this additional step, the data are generated in order to be acquired. The data are for example detected or captured (for example by an analytical device). Alternatively or additionally, the data are inputted in accordance with the additional step, for instance via interfaces. The data generated can for example be inputted (for instance into the computer). In accordance with the additional step (which precedes the acquiring step), the data can also be provided by performing the additional step of storing the data in a data storage medium (such as for example a ROM, RAM, CD and/or hard drive), such that they are ready for use within the framework of the method or program in accordance with the invention. The step of “acquiring data” can therefore also involve commanding a device to obtain and/or provide the data to be acquired. In particular, the acquiring step does not involve an invasive step which would represent a substantial physical interference with the body, requiring professional medical expertise to be carried out and entailing a substantial health risk even when carried out with the required professional care and expertise. In particular, the step of acquiring data, for example determining data, does not involve a surgical step and in particular does not involve a step of treating a human or animal body using surgery or therapy. In order to distinguish the different data used by the present method, the data are denoted (i.e. referred to) as “XY data” and the like and are defined in terms of the information which they describe, which is then preferably referred to as “XY information” and the like.

### Medical Workflow

A medical workflow comprises a plurality of workflow steps performed during a medical treatment and/or a medical diagnosis. The workflow steps are typically, but not necessarily performed in a predetermined order. Each workflow step for example means a particular task, which might be a single action or a set of actions. Examples of workflow steps are capturing a medical image, positioning a patient, attaching a marker, performing a resection, moving a joint, placing an implant and the like.

### BRIEF DESCRIPTION OF THE DRAWINGS

In the following, the invention is described with reference to the appended figures which give background explanations and represent specific embodiments of the invention. The scope of the invention is however not limited to the specific features disclosed in the context of the figures.

FIG. 1 schematically illustrates a method according to the present disclosure;

FIG. 2 schematically illustrates a medical system in which a method according to the present disclosure may be employed;

FIG. 3 illustrates biosignals from three different sweeps;

FIG. 4 illustrates another example of a biosignal;

FIG. 5 illustrates an example of a normalized biosignal;

FIG. 6 shows a Ricker Wavelet;

FIG. 7 illustrates the first derivative of a biosignal;

FIGS. 8a to 8c each illustrate an impedance signal, a CWT scalogram in the time-frequency domain, and a 3D plot CWT scalogram;

FIG. 9 shows a flowchart illustrating steps for determining whether a biosignal representative of a stimulus-induced muscle reaction is present;

FIG. 10 shows a flowchart that continues the flowchart of FIG. 9;

FIG. 11 is a schematic illustration of a system according to the present disclosure;

FIGS. 12a to 12f illustrate flowcharts of an example method.

### DESCRIPTION OF EMBODIMENTS

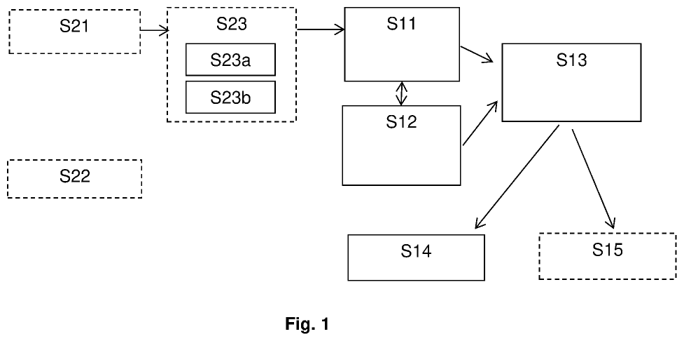

FIG. 1 illustrates the steps of a method for neuromonitoring based on a biosignal, for example an impedance signal or a bladder pressure signal, according to the present disclosure.

The method comprises the steps of monitoring and analysing a biosignal that is based on measurement data obtained by a neuromonitoring device, for example an impedance measuring device or a bladder pressure measuring device.

A portion of the biosignal may be representative of a stimulus-induced muscle reaction, particularly contraction, of smooth muscles. Optionally, the method may comprise applying a stimulus to a tissue and determining whether a muscle reaction that is stimulus-induced is detected, which implies that a nerve associated with the muscle reaction has been stimulated when applying the stimulus to the tissue.

The monitoring and analysing is performed so as to localize autonomic nerves associated with a stimulus-induced muscle reaction of smooth muscles of a target organ. That is, by applying a stimulus as explained above, it can be determined whether or not autonomic nerves are located at the location where the stimulus was applied.

The method, particularly the analysing of the biosignal, may comprise the step S11 of performing time-domain signal analysis of the biosignal to obtain one or more time-domain signal characteristics, examples of which are provided below in more detail.

The method, particularly the analysing of the biosignal, may comprise the step S12 of performing time-frequency-domain signal analysis of the biosignal to obtain one or more time-frequency-domain signal characteristics. The analysis may be performed on the biosignal itself and/or a derivative of the biosignal.

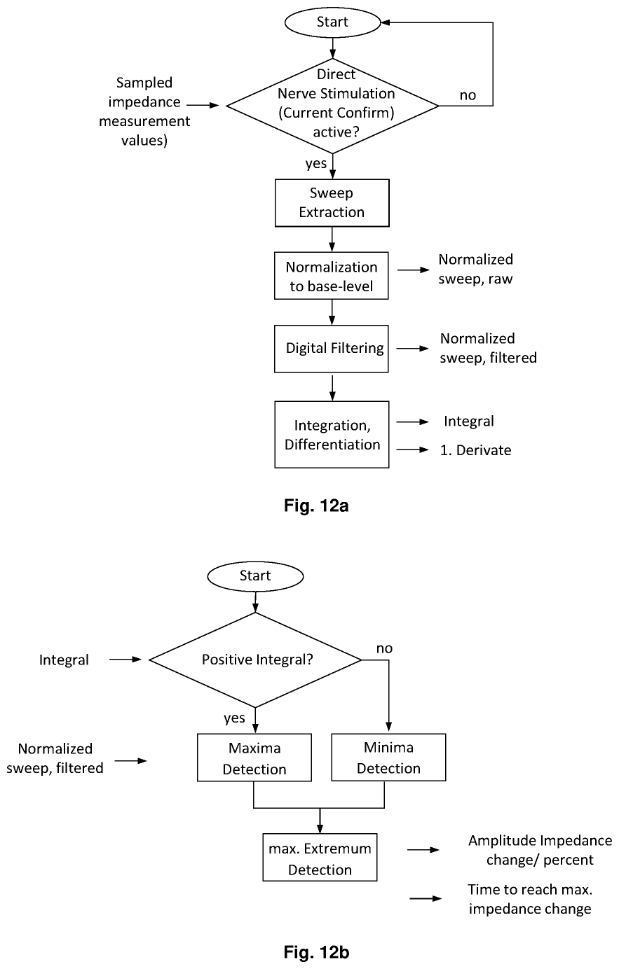

For example, a Continuous Wavelet Transform, CWT, may be performed on the biosignal or the first derivative of the biosignal. Alternatively, DWT or STFT or the like may be used. The time-frequency-domain signal analysis may comprise analysing the transformation coefficients, particularly their magnitude, as is explained in more detail in the context of FIGS. 8a to 8c.

Each of steps S11 and S12 may comprise sub-steps. Steps S11 and S12 or their sub-steps need not be performed in any particular order and may, for example, be performed consecutively or concurrently.

The method, particularly the analysing of the biosignal, may comprise the step S13 of, based on the time-domain signal characteristics and time-frequency-domain signal characteristics determining whether the biosignal is representative of a stimulus-induced muscle reaction.

Criteria may be associated with the shape of the biosignal. Determining whether the biosignal is representative of a stimulus-induced muscle reaction may entail distinguishing between features of the biosignal representative of a stimulus-induced muscle reaction, artifacts, or features of the signal that do not represent a significant biosignal change.

The method of the present disclosure comprises the step S14 of outputting an indication that a stimulus-induced muscle reaction has been detected in case it is determined that the biosignal is representative of a stimulus-induced muscle reaction. For example, a visual and/or audio output may be provided to a user. The output may serve as an indicator that the portion of the tissue to which the stimulus is/was applied contains nerves associated with the smooth muscles of the target organ. Accordingly, autonomic nerves can be localized.

Optionally, in step S15, in case no stimulus-induced muscle reaction has been detected, the method may comprise outputting an indication that no stimulus-induced muscle reaction has been detected. The output may serve as an indicator that the portion of tissue to which the stimulus was applied does not contain nerves associated with the smooth muscles of the target organ.

In optional step S21 measurement data is acquired by means of a neuromonitoring device. The measuring may comprise measuring an impedance of smooth muscles of a target organ by means of an impedance measurement device or measuring a bladder pressure by means of a pressure measurement device, for example.

The measuring may be performed continuously. The measurement data may be used as the biosignal and, accordingly, as input data for analysing the biosignal. Alternatively, the measurement data may be pre-processed so as to obtain the biosignal, which is then used as input data for analysing the biosignal.

In optional step S22, a stimulus is applied to a portion of a tissue. Optionally, this may trigger the monitoring and/or the analysing of the biosignal. Alternatively the monitoring and/or the analysing may be performed irrespective of whether a stimulus is applied.

In optional step S23, the measurement data obtained by step S21 is pre-processed so as to obtain the biosignal, e.g., an impedance signal or a bladder pressure signal. Step S23 is shown as comprising the pre-processing step of normalizing the signal S23a, e.g., with respect to a base-level or base-value of the biosignal, and the pre-processing step of applying a filter S23b, e.g., a low pass filter.

For the sake of completeness, it is noted that the steps outlined above may be performed for one target organ or for two or more target organs, each having a corresponding biosignal. In this case, the signal processing and analysis may be performed for each of the biosignals separately in the manner described above.

This does not require applying separate stimuli to the tissue. In particular, in a preferred embodiment, a common stimulus may be used and each of two or more biosignals may be analysed to determine whether it is representative of a stimulus-induced muscle reaction.

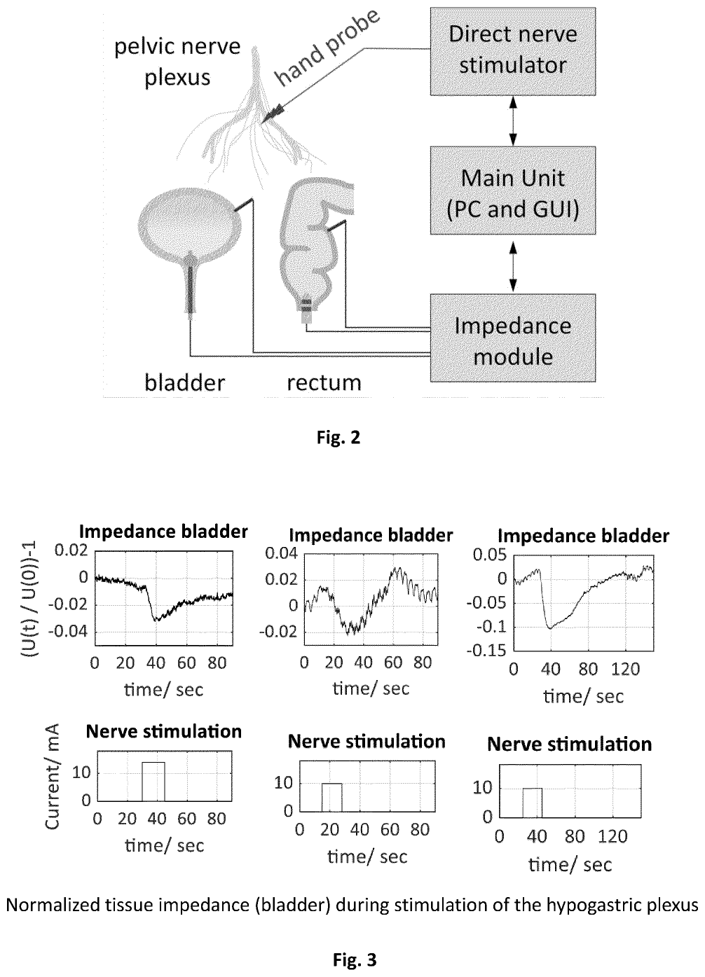

FIG. 2 illustrates an exemplary medical system which may be used to carry out method according to the present disclosure, as well as exemplary organs and nerves. In the figure, pelvic nerves and the hand probe are schematically shown. Moreover, a bladder and rectum with impedance measurement electrodes of an impedance measurement device connected to each organ are shown. In this example, the impedance measurement device is referred to as an impedance module and it is in communication with the computing system, which is referred to as a main unit. The main unit may comprise a data processing component and a graphical user interface. Moreover, connected to the main unit is direct nerve stimulator for applying a stimulus to nerves, which may comprise a hand probe to contact the tissue for applying the stimulus.

As an example, pelvic nerves are shown in this figure. As schematically implied, the nerves are very fine and difficult to identify optically, such that the impedance-based identification of the presence of a nerve is advantageous.

In the following, examples for some of the above methods steps are presented in more detail. It is noted that the following steps are described using an example wherein the biosignal is an impedance signal. However, the alternatively the biosignal may be a bladder pressure signal or some other signal representative of a stimulus-induced muscle reaction of smooth muscles of a target organ. The steps described below may be applied in the described manner to those alternative biosignals as well.

Moreover, the following discussion is based mostly on the use of CWT for performing time-frequency domain signal analysis. However, it is to be understood that DWT or STFT may alternatively be used and that the analysis can be performed in an analogous manner when using DWT or STFT by analysing their respective transformation coefficients in a manner similar to the one described below for the CWT transformation coefficients.

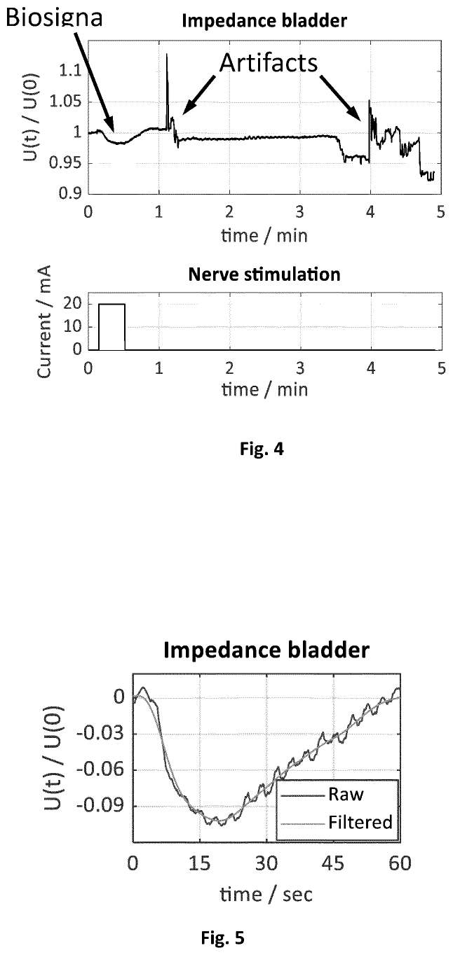

FIG. 3 illustrates biosignals, in this example impedance signals, from three different sweeps. Each of the biosignals is representative of a stimulus-induced muscle reaction. In this figure, normalization has already been performed on the impedance measurement data. As can be seen from the figure, the impedance has been normalized with respect to a base-level impedance. The figure also illustrates the respective stimulus applied to the nerve. In the present example the stimulus is a rectangular wave applied at different times and with different amplitudes.

It can be seen from FIG. 3 that the shape of the impedance curve for each of the stimuli is similar, such that, in general, the shape of the curve is a good predictor for determining whether nerve stimulation has occurred. However, as can also be seen from the figure, there are various factors that make recognizing the shape difficult, particularly for a human visually reviewing the impedance signal. Accordingly, the automatic signal analysis method of the present disclosure provides improved reliability.

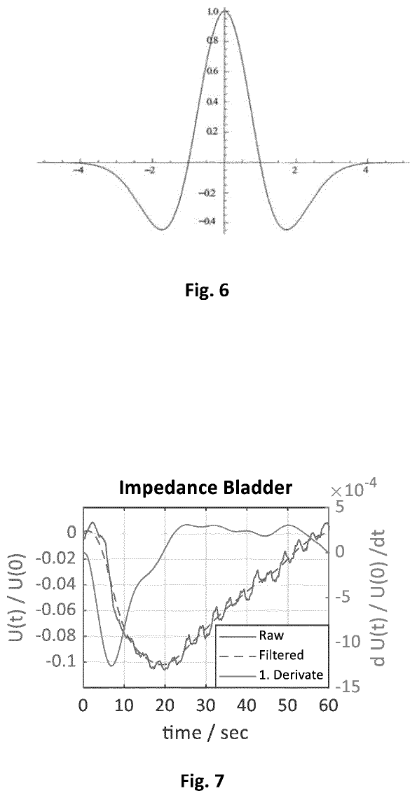

FIG. 4 illustrates another example of an impedance signal that includes both, artifacts and impedance changes due to stimulation of a nerve. It becomes apparent that artifacts may be a big problem for properly analysing the impedance signal, such that the signal analysis steps of the present disclosure, which allow for distinguishing between biosignals representative of stimulus-induced muscle reactions and artifacts, are particularly advantageous.

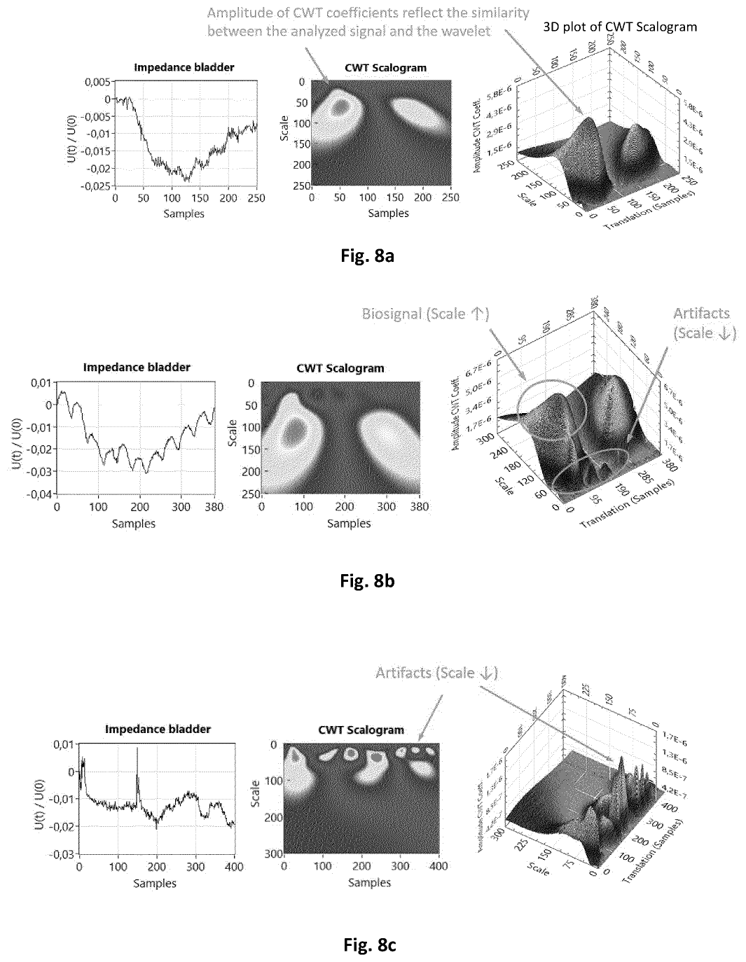

FIG. 5 illustrates an example of a normalized impedance signal, in this example of the impedance of the bladder, and a filtered impedance signal. In this example, a third order IIR Bessel low pass filter with an exemplary frequency of 0.15 Hz was used. However, different filters and parameters may be used instead.

Regarding the time-frequency signal analysis, as explained previously, a STFT is one possibility for transforming the impedance signal into the time-frequency domain. However, for improved frequency and time resolution, the Continuous Wavelet Transform (CWT) may be used instead.

The Continuous Wavelet Transform of a function is determined by the inner product of the function and the wavelet family, multiplied by a normalization factor, and can be represented by the following equation

$$C W T ( k , a ) = \frac{1}{\sqrt{a}} ⋆ \int x ( t ) * Ψ ( \frac{t - k}{a} ) d t .$$

The first term 1/√a represents the normalization factor to normalize between different wavelet scales, with a being the scaling factor of the wavelet.

The second term ∫x(t) represents the analysed function, in the present disclosure the analysed biosignal. As the CWT is applied to sampled measurement data, the method of the present disclosure is a discretized CWT.

The third term

$$Ψ ( \frac{t - k}{a} )$$

represents the wavelet family, which includes a prototype wavelet (mother wavelet) and scaled and shifted versions of the mother wavelet. Wavelets are window functions of oscillating character with finite duration. Scaling includes stretching and compression of the wavelet in time, whereby stretching the wavelet (large scaling factors) results in reducing its original frequency, and compression of the wavelet (small scaling factors) results in increasing its original frequency.

In the above equation, k represents a shifting factor. Shifting of the mother wavelet and its scaled versions results in coefficients indicating the similarity between the analysed signal and the wavelet, dependent on the frequency (scaling factor) and time (shifting factor).

There is a reciprocal relationship between the wavelet scale and the equivalent frequency, with a constant of proportionality. The equivalent/pseudo frequency F_{pseudo} is represented by the following equation

$$Fpseudo = \frac{F c}{a * Δ t} = \frac{F c * f s}{a}$$

F_{c} represents the centre frequency of the mother wavelet (which is the constant of proportionality) and Fₛ represents the sampling frequency, and Δt the sampling interval.

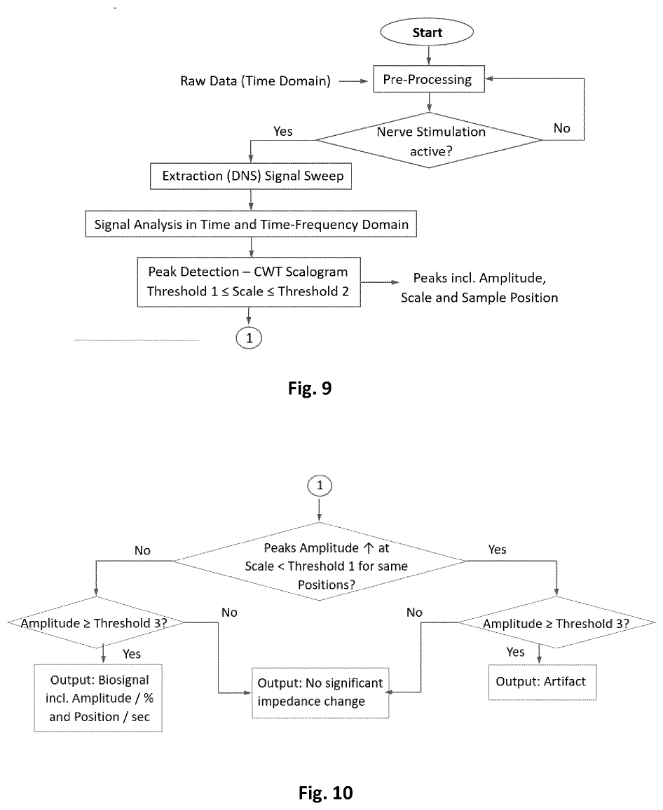

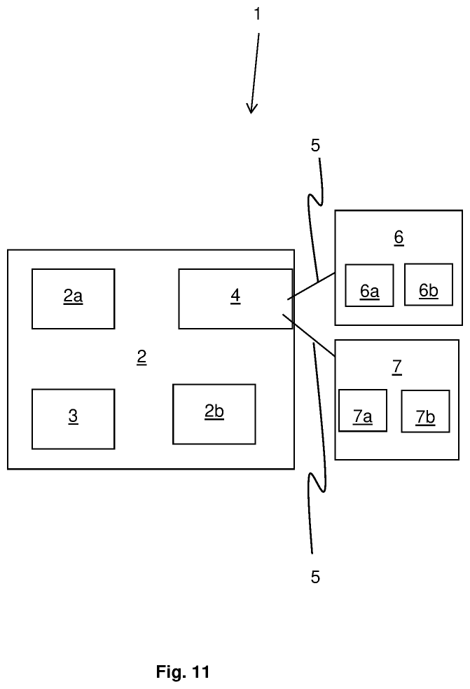

FIG. 6 shows a so-called Ricker Wavelet, also referred to as Mexican Hat Wavelet, which has a signal shape that is similar to the first derivative of the impedance signal as shown in FIG. 7, which is representative of a stimulus-induced muscle reaction. In FIG. 7, a normalized signal (labelled “raw”), and a normalized and filtered signal (labelled “filtered”) and its first derivate are shown.

FIGS. 8a to 8c each show an impedance signal, a CWT scalogram which reflects the magnitude of the CWT coefficients which corresponds to the time-frequency representation of the impedance signal, and a 3D plot CWT scalogram.

In FIG. 8a, as can be seen in the impedance signal, an impedance change occurs. The scalogram of the time-frequency representation of the impedance signal shows that maxima in the CWT coefficients magnitude (also referred to in the following as “peaks”) can be found in two regions. This can also be seen in the form of two peaks in the 3D plot. Both peaks may be candidate features that could be representative of a stimulus-induced muscle reaction and the resulting impedance change.

FIG. 8b shows candidate features similar to the ones shown in FIG. 8a, such that a stimulus-induced muscle reaction may be identified in the biosignal based on said peaks. In addition, maxima with a lower magnitude (smaller peaks) are visible in the 3D plot. These smaller peaks may represent artifacts and be discarded from a set of candidate features that might be representative of a stimulus-induced muscle reaction.

In general, larger scaling factors may be indicative of a feature being representative of a stimulus-induced muscle reaction, whereas scaling factors being lower may be indicative of a feature being representative of artifacts.

FIG. 8c shows various maxima in the CWT coefficients magnitude at lower wavelet scaling factors, particularly peaks may be representative of artifacts. Accordingly, this impedance signal of FIG. 8c would be identified as not being representative of a stimulus-induced muscle reaction.

As can be seen from the above, extrema in the CWT coefficients magnitude (peaks) at lower wavelet scales may be representative of artifacts and peaks at larger wavelet scales may be representative of a stimulus-induced muscle reaction. A predetermined threshold of the wavelet scale (corresponding to the maxima in the CWT coefficients magnitude) may be the primary criterion to distinguish between stimulus-induced muscle reactions and artifacts. The threshold for the scale separating candidate features representative of artifacts from candidate features representative of stimulus-induced muscle reactions may be determined, for example, based on a plurality of control data.

Alternatively, or in addition, candidate features, for example maxima in the CWT coefficients magnitude, may be discarded as artifacts or non-significant impedance changes if the magnitude of the feature is below a predetermined threshold.

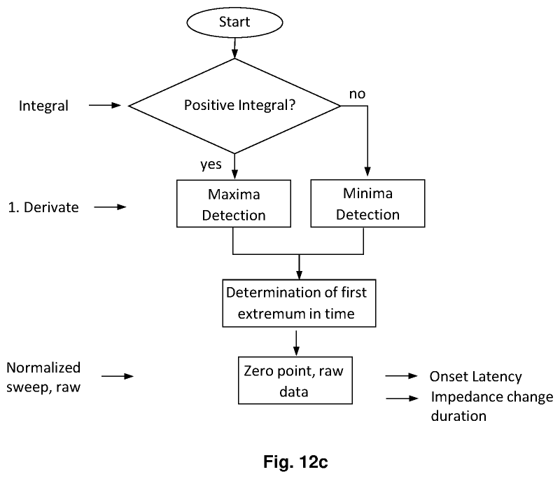

FIG. 9 shows a flowchart for determining whether a biosignal representative of a stimulus-induced muscle reaction is present. At the beginning raw data in the time domain is input and pre-processing is performed. In a first step, it may be determined whether nerve stimulation is active. If not, no determination is necessary. The method may return to the pre-processing. If direct nerve stimulation (DNS) is active, an optional signal sweep extraction is performed. A signal analysis is performed in the time domain and the time-frequency domain for example as explained above. Maxima in the CWT coefficients magnitude (peaks) are detected for wavelet scales exceeding a predetermined threshold (Threshold 1, e.g. 37) and remaining below a predetermined threshold (Threshold 2, e.g. 300). Thus, candidate peaks including magnitude, wavelet scale, and time relation are obtained, which may be representative of the stimulus-induced muscle reaction and a corresponding impedance change. However, some of the candidate peaks may not be representative of the stimulus-induced muscle reaction, but rather of artifacts or insignificant impedance changes.

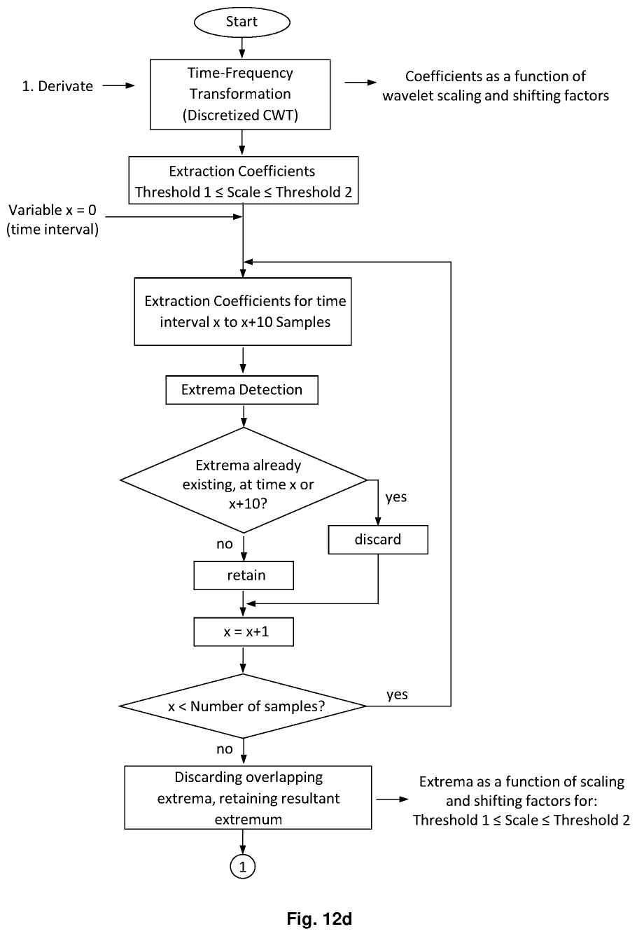

FIG. 10 shows a flowchart that continues the previous flowchart to illustrate such a categorization. Therefore, maxima in the CWT coefficients magnitude (peaks) are detected for wavelet scales below a predetermined threshold (Threshold 1). Thus, candidate peaks including magnitude, wavelet scale, and time relation are obtained, which may be representative of artifacts.

Resultant candidate peaks with corresponding wavelet scale below the predetermined threshold wavelet scale (Threshold 1) and resultant candidate peaks with corresponding wavelet scale above the predetermined threshold wavelet scale (Threshold 1), that fall within a predetermined time interval, particularly overlapping candidate peaks, may be compared in magnitude. Candidate peaks with corresponding wavelet scale below the predetermined threshold wavelet scale and with corresponding magnitude below the magnitude of the candidate peak with corresponding wavelet scale above the predetermined threshold wavelet scale are discarded, indicating a candidate peak representative of the stimulus-induced muscle reaction.

Candidate peaks with corresponding wavelet scale below the predetermined threshold wavelet scale and with corresponding magnitude larger than the magnitude of the candidate peak with corresponding wavelet scale above the predetermined threshold wavelet scale are indicating a candidate peak representative of an artifact.

Additionally, it is determined whether the maximal amplitude within the biosignal waveform in time domain is not bigger than the Threshold 3 (e.g., for example, be 0.9%) and, if so, no significant impedance change occurred.

Thresholds 1 to 3 may be predetermined threshold values obtained empirically or semi-empirically.

For the sake of completeness, it is noted that the method has been tested already successfully in an animal study and in clinical investigations with humans. The studies have shown that using a signal analysis in the time domain and the time-frequency domain according to the present this closure, e.g. using discretized CWT of a first derivative of the biosignal and a Ricker Wavelet, results in signal features which enable differentiation between biosignals representative of a stimulus-induced muscle reaction and artifacts.

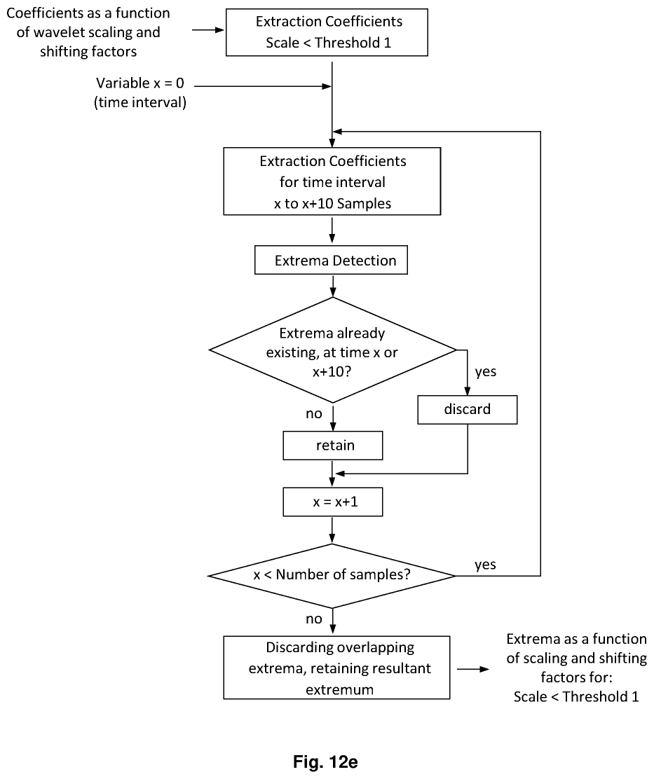

In FIG. 11, a schematic illustration of a medical system 1 according to the present disclosure is shown. The medical system may comprise a computing system 2 comprising, for example, processing means 2a and storage means 2b, which may comprise temporary memory, e.g., RAM, and/or permanent memory, e.g., ROM. The computing system may also comprise a display device 3 or be connected to a display device. Moreover, optionally the computing system may comprise one or more communication interfaces 4 for receiving and transmitting data via one or more data connections 5. For example, the computing system may comprise one or more computers.

The medical system may further comprise a neuromonitoring device 6 configured to acquire measurement data. In particular, the neuromonitoring device may be configured to, particularly continuously, measure an impedance of smooth muscles of a target organ, e.g., bladder or rectum, or a bladder pressure. The neuromonitoring device, e.g., the impedance measurement device, may be connected to the computing system via one of the communication interfaces 4. Alternatively, the processing means of the computing system may at least in part be comprised in the neuromonitoring device. The impedance measurement device may comprise two electrodes 6a and 6b attachable to a tissue, particularly a smooth muscle of a target organ.

The medical system may further comprise a, particularly hand-held, device 7 configured to apply a stimulus to a portion of a tissue. For example, the device may comprise an electrode 7a and a power supply 7b. The device 7 may be connected to the computing system via one of the communication interfaces 4 and may be configured to provide, to the computing system, information indicating the timing and/or characteristics of a stimulus being applied.

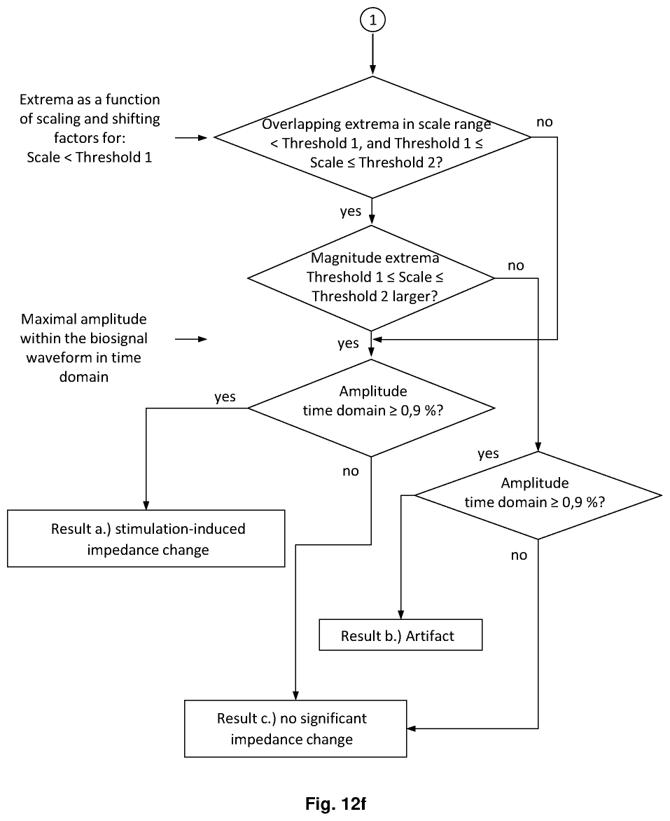

An even more detailed example of the method of the present disclosure is provided below and illustrated in the flowcharts shown in FIGS. 12a to 12f.

### EXAMPLE

Changes in the impedance signal triggered by a slow contraction of smooth muscles during a direct stimulation of innervated nerves are characterized by a characteristic shape of the signal. After applying the stimulation for a few seconds to nerve tissue, a positive or a negative change of impedance is observed, which peaks after a few seconds. After reaching the maximum, i.e., peaking, and after discontinuing the nerve stimulation, a relaxation phase of several seconds is performed until an initial level is restored. Stimulations of the nerves of different strengths and, accordingly, contractions of different strengths lead to differences in the maximum signal amplitude and the gradient of the change, i.e., the frequency of the biosignal. The characteristic shape of the signal is conserved.

According to the present disclosure, an automatic, software-based analysis of the impedance signal may be performed so as to detect characteristic impedance changes induced by stimulation of autonomic nerves and resulting contraction of smooth muscles, particularly so as to discriminate artifacts. Signal analysis is performed subsequently to direct nerve stimulation. The signal analysis comprises a classification with information at least on the existence of characteristic changes in impedance induced by stimulation, existence of an artifact, or no significant impedance change. The classification of the reaction to stimulation and, accordingly, distinguishing artifacts from physiologically induced positive stimulation reactions or no significant impedance changes is performed based on signal characteristics in the time domain and in the time-frequency domain.

Below exemplary steps of a method according to the present disclosure are presented in detail.

### Data Pre-Processing

In the following, an example for data pre-processing, which may for example be employed in step S23 of FIG. 1, will be presented in detail. It is noted that this merely serves as an illustrative example and other pre-processing steps may be performed.

Signal analysis of the impedance signal at target organs, e.g., bladder and/or rectum, is performed at least from the time of applying a direct nerve stimulation. This is also referred to as current confirmed phase. It may be the time in which a stimulation pulse is applied to the tissue. A sampling frequency of 10 Hz may be used. In particular 50 data points that correspond to five seconds of data acquisition are collected for each impedance measurement channel prior to signal analysis.

The change of tissue impedance during muscle contraction is evaluated compared to the state prior to contraction. To do so, the signal portions, also referred to as sweeps, extracted for analysis are normalized to the impedance level prior to contraction, which is also referred to as initial-level or base-level impedance, also referred to as base impedance. The base impedance may correspond to the impedance of the connection of the main device, current leads, electrodes, and tissue. It may be determined by calculating the mean U(0) of a predetermined number of samples, for example 15 samples, at the beginning of the stimulation. The extracted sweeps are normalized by dividing the value of each sample within the sweep by the determined mean U(0), e.g., of the first 15 samples. After subtraction of 100% (value 1), a dimensionless signal shape (U(t)/U(0)−1) is obtained, which is proportional to the change of the tissue impedance during muscle contraction.

The normalized sweeps are objected to a low pass filter, so as to suppress superimposing of signals like spontaneous changes of the membrane potential, noise, or artifacts caused by respiration. Moreover, the low pass filter smooths the signal. Low pass filtering may be performed, e.g., using a digital IIR filter, such that the characteristic shape of the impedance change, which was caused by contraction of smooth muscles, remains essentially unchanged.

Subsequently, the integral and the first derivative, i.e. the shape of the gradient, of the normalized and low pass filtered sweep may be determined. They may be input parameters for signal analysis in the time domain and in the time-frequency domain.

### Signal Analysis in the Time Domain

In the following, an example for time domain signal analysis, which may for example be employed in step S11 of FIG. 1, will be presented in detail. It is noted that this merely serves as an illustrative example and other analysis steps may be performed.

The signal analysis in the time domain may comprise determining at least one of the following:
a maximum amplitude of the impedance change, for example in percent relative to the initial level, i.e., base impedance
onset latency of the signal.
duration of the impedance change, e.g., from the onset of the impedance change up to the maximum impedance change

Depending on the result of the previously determined integral value, local extrema of the signal, e.g., minimum of a negative integral value or maximum of a positive integral value, are detected.

For example, a threshold value for determining local extrema may be 25% of the maximum value within the sampling interval of the sweep being analysed. As such, multiple local extrema in this sampling interval are possible. The maximum extrema, e.g., the maximum or minimum, after onset of the application of the direct nerve stimulation (current confirmed phase) correspond to maximum amplitude of impedance change in percent relative to the initial level. Impedance changes with an amplitude below a certain threshold, e.g. 0.9%, are not characterized as significant impedance changes.

The onset latency corresponds to the time from initiating application of the direct nerve stimulation (current confirmed phase) until onset of the impedance change. For calculating the onset latency, local extrema of the first derivative of the signal are determined. The first local extrema on the perspective of time of the first derivative is determined, i.e., the first maximum gradient. By a backwards search starting from the previously determined point in time, the zero transition in the normalized and unfiltered raw signal is determined. This value corresponds to the onset latency of the impedance change. The onset latency may be output as a parameter for monitoring by a user.

The duration of impedance change may be determined by subtracting the onset latency from the time until maximum amplitude of the impedance change is reached.

### Signal Analysis in the Time-Frequency Domain

In the following, an example for time-frequency-domain signal analysis, which may for example be employed in step S12 of FIG. 1, will be presented in detail. It is noted that this merely serves as an illustrative example and other analysis steps may be performed.

For determining signal features that can be used for distinguishing between a stimulus-induced positive stimulation reaction and artifacts that are caused by non-stimulus-induced organ movements, data are transformed into the time-frequency domain. Due to the time frequency transformation, frequency information of the signal is determined for each point in time, whereby characteristic transient signal changes of nonstationary signals can be determined. Such signal changes may comprise the onset of an impedance change at the target muscle during stimulation of innervated nerves.

The first derivative of the normalized filtered sweep may be transformed to the time frequency domain by means of discretized continuous wavelet transform (CWT). This corresponds to a convolution of the sweep with a time-limited window function of oscillating character (wavelet), which allows for decomposition of the signal in a time domain and the frequency domain. The convolution is repeated iteratively for time-shifted wavelets (using shifting factors) and frequency-changed wavelets (using scaling factors). This results in a function of transformation coefficients as a function of time and the equivalent frequency. Compression and stretching of the mother wavelet (initial function/prototype for all window functions), which are summarized as scaling of the wavelet, are inversely proportional to the equivalent frequency, such that the detected equivalent frequency decreases with increased scaling factor (wavelet stretching) and increases with decreasing scaling factor (wavelet compression). The time resolution is proportional to the shifting (which may also be referred to as translation) of the wavelet and improves with smaller shifting factor. The higher the absolute magnitude of the resulting coefficient at a predetermined point in time, the higher the correlation of the wavelet with the analysed signal at this time.

In order to allow for signal analysis with as few data points as possible at the earliest possible point in time, characteristic impedance change is detected immediately after onset and preferably prior to reaching the maximum. This earliest possible point in time, in practice, may mean as soon as possible after the direct nerve stimulation onset. Due to its similarity with the signal shape of the first derivative of a biosignal representative of a stimulus-induced muscle reaction and corresponding impedance change, preferably the Ricker Wavelet (Mexican Hat Wavelet) is used. Scaling and shifting parameters are preferably set to 300 scalings and stepwise shifting of a data point. From the transformation, coefficients that depend on time and wavelet scaling (proportional to frequency) are obtained. The coefficients can be represented in an intensity graph (scalogram).

Subsequently, extrema in the three-dimensional data space of the magnitude of the coefficients resulting from the transformation are detected. The detection is performed preferably in a window of 10 data points which, is iteratively moved along the time axis starting at zero up until the maximum number of data points in increments/stepwise. The threshold for detecting an extremum is adjusted after shifting the window and may for example be 25% of the maximum magnitude of the coefficients within the window.

Extrema within the window are preferably detected within a scaling interval bigger than or equal to a threshold, e.g., 37 for a scanning rate of 10 Hz and a frequency of the mother wavelet of 0.25 Hz. Extrema at the left and right boundaries of the window and extrema that correspond to already detected extrema may be discarded. As a result, extrema are obtained dependent on the corresponding scaling coordinate and shifting coordinate. The magnitude of the coefficients of the transformation can be visualized for the detected extrema dependent on time in two dimensions.

Subsequently, the smoothing of the detected extrema dependent on time is performed, which may correspond in this case to summarizing extrema that are close together on a time axis. As a threshold, one third of the mean expected duration of a characteristic impedance change, e.g., in data points, can be used, for example a duration of 61 data points. In case several extrema having a time distance of less data points than the threshold are detected, the maximum extremum of the local extrema located next to each other and within this time is determined and all other extrema are discarded. This results in smoothed extrema dependent on the corresponding scaling coordinate and shifting coordinate for the scaling region of equal to or more than a threshold, e.g., 37.

Subsequently, the detection of extrema in the three-dimensional data space of the magnitude of the coefficients resulting from the transformation is repeated in the scaling region of less than the threshold, e.g., 37 for a sampling frequency of 10 Hz and the frequency of mother wavelet of 0.25 Hz, with the same parameters and steps as previously described above. This results in smoothed extrema in dependency of the corresponding scaling coordinate and shifting coordinate for the scaling region of less than the threshold, e.g., 37.

In case there is, in a window region of the previously defined threshold (for example 61 data points), an extremum in both scaling regions, the magnitude of these extrema, which corresponds to the magnitude of the coefficient of the transformation, is compared. This allows for differentiating of stimulus-induced characteristic impedance changes and non-stimulus-induced impedance changes and/or artifacts. In case the magnitude of an extremum in the scaling region of above the threshold, e.g. 37, is bigger than the magnitude of the extremum in the scaling region of less than the threshold, e.g. 37, a stimulus-induced characteristic impedance change is likely present. Otherwise, an artifact is likely present. If the amplitude determined for the period of time within the sweep is less than a threshold, e.g., 0.9%, the result of the signal analysis may be that there is no significant impedance change.

While the invention has been illustrated and described in detail in the drawings and foregoing description, such illustration and description are to be considered exemplary and not restrictive. The invention is not limited to the disclosed embodiments. In view of the foregoing description and drawings it will be evident to a person skilled in the art that various modifications may be made within the scope of the invention, as defined by the claims.

1. A method for neuromonitoring based on a biosignal, the method comprising:
monitoring and analysing the biosignal for localizing autonomic nerves associated with a stimulus-induced muscle reaction of smooth muscles of a target organ, by:
performing time-domain signal analysis of the biosignal to obtain one or more time-domain signal characteristics, the biosignal based on measurement data obtained by a neuromonitoring device;
performing time-frequency-domain signal analysis of the biosignal to obtain one or more time-frequency-domain signal characteristics; and
determining, based on the time-domain signal characteristics and the time-frequency-domain signal characteristics-determining, the biosignal is representative of the stimulus-induced muscle reaction; and
outputting an indication that the stimulus-induced muscle reaction has been detected based on determining that the biosignal is representative of the stimulus-induced muscle reaction.

2. The method of claim 1, further comprising:
based on the time-domain signal characteristics and the time-frequency-domain signal characteristics, distinguishing between features in the biosignal that are representative of artifacts and features in the biosignal that are representative of the stimulus-induced muscle reaction, and
optionally, distinguishing between features in the biosignal that are not representative of a significant biosignal response.

3. The method of claim 1, wherein the obtained one or more time-domain signal characteristics comprise at least one of the following:
a maximum amplitude within the biosignal relative to a base-level of the biosignal, the biosignal being a waveform,
an onset latency of a transient signal change within the biosignal,
a gradient of a transient signal change within the biosignal,
a time to reach maximum gradient of a transient signal change within the biosignal,
a duration of a transient signal change within the biosignal from an onset of the transient signal change, and/or
regression coefficients.

4. The method of claim 1, wherein the obtained one or more time-frequency-domain signal characteristics comprise a magnitude of transformation coefficients.

5. The method of claim 1, wherein the performing the time-frequency-domain signal analysis comprises:
transforming the biosignal or a first derivative of the biosignal to the time-frequency-domain; and
analysing one or more transformation coefficients,
optionally, the analysing comprises analysing one or more transformation coefficients of a Wavelet Transform (WT) or of a Short-Time Fourier Transform (STFT).

6. (canceled)

7. The method of claim 5, wherein the performing the time-frequency-domain signal analysis comprises:
selecting a window function and scaling the window function;
obtaining, for each of a plurality of samples, one or more transformation coefficients; and
analysing at least a subset of the one or more transformation coefficients.

8. The method of claim 5, wherein the analysing the one or more transformation coefficients comprises:
representing a magnitude of one or more of the transformation coefficients as a function of time and frequency;
identifying candidate features in the function; and
determining, for at least one of the identified candidate features, the candidate feature is representative of the stimulus-induced muscle reaction, the determining based on one or more of the time-domain signal characteristics and/or based on one or more of the time-frequency-domain signal characteristics.

9. The method of claim 5, wherein the performing the time-frequency-domain signal analysis comprises analysing the one or more transformation coefficients taking into account the one or more time-domain signal characteristics and/or information derived from the one or more time-domain signal characteristics.

10. The method of claim 8, wherein the performing the time-frequency-domain signal analysis comprises:
determining a time frame corresponding to a selected portion; and
determining whether a candidate feature is representative of the stimulus-induced muscle reaction at least based on the determined time frame.

11. The method of claim 1, the method comprising any one of the following:
performing a Continuous Wavelet Transform (CWT) of the biosignal or a first derivative of the biosignal, or
performing a Discrete Wavelet Transform (DWT) of the biosignal or a first derivative of the biosignal, or
performing a Short-Time Fourier Transform (STFT) of the biosignal or a first derivative of the biosignal.

12. The method of claim 1, wherein the monitoring and analysing of the biosignal is triggered by receiving an indication of a stimulus being applied to a tissue, and/or wherein the time-domain signal analysis and/or the time-frequency-domain signal analysis are based on one or more characteristics of the stimulus being applied to the tissue.

13. The method of claim 1, further comprising:
pre-processing an output signal of the neuromonitoring device to obtain the biosignal, the pre-processing comprising any one or more of the following:
normalizing the output signal with respect to a base-level of the biosignal,
applying a low pass filter, and/or
performing a sweep extraction.

14. The method of claim 1, further comprising:
integrating and/or differentiating the biosignal, wherein results of the integrating and/or the differentiating are used as input for the time-domain signal analysis and/or for the time-frequency-domain signal analysis.

15. The method of claim 14, comprising:
performing continuously or for each of a plurality of sweeps of the biosignal, the time-domain signal analysis and the time-frequency-domain signal analysis, and
optionally, performing continuously or for each of a plurality of sweeps of the biosignal, the integrating and/or the differentiating of the biosignal.

16. (canceled)

17. The method of claim 1, wherein the biosignal is an impedance signal or a bladder pressure signal, and/or wherein the stimulus-induced muscle reaction is a muscle contraction.

18. (canceled)

19. The method of claim 1, further comprising:
acquiring, by the neuromonitoring device, measurement data;
applying a stimulus to a portion of a tissue; and
in response to determining that the biosignal is representative of the stimulus-induced muscle reaction caused by applying the stimulus to the portion of the tissue, outputting an indication that the portion of the tissue comprises nerves associated with the smooth muscles.

20. The method of claim 1, further comprising:
performing the monitoring and analysing and the outputting steps for at least two biosignals, the biosignals obtained for different target organs for localizing autonomic nerves associated with the stimulus-induced muscle reaction of smooth muscles of each of the different target organs.

21. A medical system, comprising:
a computing system, the computing system comprising:
at least one computer comprising a non-transitory memory device and at least one processor configured to communicate with the non-transitory memory device, the non-transitory memory device storing a computer program comprising executable instructions that, when executed on the at least one processor of the at least one computer or loaded onto the at least one processor of the at least one computer, cause the at least one computer to perform a method for neuromonitoring based on a biosignal by:
monitoring and analysing the biosignal for localizing autonomic nerves associated with a stimulus-induced muscle reaction of smooth muscles of a target organ, by:
performing time-domain signal analysis of the biosignal to obtain one or more time-domain signal characteristics, the biosignal based on measurement data obtained by a neuromonitoring device;
performing time-frequency-domain signal analysis of the biosignal to obtain one or more time-frequency-domain signal characteristics; and
determining, based on the time-domain signal characteristics and time-frequency-domain signal characteristics, the biosignal representative of the stimulus-induced muscle reaction; and
outputting an indication that the stimulus-induced muscle reaction has been detected based on determining that the biosignal is representative of the stimulus-induced muscle reaction.

22. The medical system of claim 21, further comprising:
a device operatively connected to the computing system and configured to apply a stimulus to a portion of a tissue, and optionally, the device is configured to provide, to the computing system, information indicating timing and/or characteristics of the stimulus being applied; and/or
the neuromonitoring device operatively connected to the computing system and configured to acquire the measurement data; and/or
wherein the outputting comprises outputting, by an output device of the computing system, the indication that the stimulus-induced muscle reaction has been detected, and optionally, wherein the output device comprises at least one of a visual output device, an audio output device, or a haptic output.

23. (canceled)

24. A non-transitory computer-readable storage medium storing a computer program comprising program instructions that, when executed on at least one processor of a computer or loaded onto the at least one processor of the computer, cause the computer to perform a method for neuromonitoring based on a biosignal by:
monitoring and analysing the biosignal for localizing autonomic nerves associated with a stimulus-induced muscle reaction of smooth muscles of a target organ, by:
performing time-domain signal analysis of the biosignal to obtain one or more time-domain signal characteristics, the biosignal based on measurement data obtained by a neuromonitoring device;
performing time-frequency-domain signal analysis of the biosignal to obtain one or more time-frequency-domain signal characteristics; and
determining, based on the time-domain signal characteristics and time-frequency-domain signal characteristics, the biosignal is representative of the stimulus-induced muscle reaction; and
outputting an indication that the stimulus-induced muscle reaction has been detected based on determining that the biosignal is representative of the stimulus-induced muscle reaction.
